(19) United States
(12) Patent Application Publication
(10) Pub. No.: US 2019/0298273 A1
(43) Pub. Date: Oct. 3, 2019

(54) System For Recognizing Operator Attention And Preventing Diversion

(71) Applicant: Bilateral, Inc., Philadelphia, PA (US)

(72) Inventor: Jacob Strupp, Philadelphia, PA (US)

(73) Assignee: Bilateral, Inc., Philadelphia, PA (US)

(21) Appl. No.: 16/372,891

(22) Filed: Apr. 2, 2019

Related U.S. Application Data
(60) Provisional application No. 62/651,834, filed on Apr. 3, 2018.

(51) Int. Cl. A61B 5/00; G01P 15/097; G01P 21/00; G06F 3/01; G08B 21/06; A61B 5/18; A61B 5/053

(52) U.S. Cl. CPC A61B 5/746; G01P 15/097; G01P 21/00; G06F 3/013; A61B 2560/0223; A61B 5/18; A61B 5/053; A61B 5/7282; G08B 21/06

(57) ABSTRACT

A system for monitoring attentiveness of an operator, having a data acquisition unit and a control unit. The data acquisition unit is adapted to be worn by the operator, and includes a capacitive electrode configured to detect electrical activity induced by eye blinks of the operator, a converter circuit configured to process the detected electrical activity into a digital signal, and a wireless transmitter configured to wirelessly transmit the digital signal. The control unit is positioned remote from the data acquisition unit, and includes a wireless receiver configured to receive the digital signal from the wireless transmitter, and a control circuit communicatively coupled to the wireless receiver to receive the digital signal, and configured to identify a plurality of eye-closing events and a plurality of eye-opening events from the digital signal, and determine a state of attentiveness of the operator based on the identified eye-closing and eye-opening events.

### CROSS-REFERENCE TO RELATED APPLICATIONS

The present application claims the benefit of the filing date of U.S. Provisional Patent Application No. 62/651,834 filed Apr. 3, 2018, the disclosure of which is hereby incorporated herein by reference.

### TECHNICAL FIELD OF THE INVENTION

The present application relates to operator awareness systems, and particularly to systems that monitor an operator's awareness and proactively prevent diversions to the operator.

### BACKGROUND

Vehicle operators encounter many distractions when driving, such as from mobile devices, built-in vehicle consoles, and fellow passengers. Additionally, a vehicle operator may be drowsy while driving. Distractions and drowsiness can impair the operator's focus, potentially making operation of the vehicle unsafe.

In order to improve safety conditions for vehicle operators, it has been proposed to monitor the operator's attentiveness by tracking the operator's eye movements. Such tracking commonly uses an infrared detector positioned across from the operator's face to detect motion. However, atmospheric conditions in the vehicle can make infrared detection difficult and thus unreliable. It is therefore desirable to track eye movements of the vehicle operator using other more reliable means.

Vehicle operators are also encouraged or even warned to turn off or put away mobile devices while driving. However, not all operators heed these warnings. It is further desirable to ensure that vehicle operators cannot access their mobile devices or other distracting items while operating the vehicle, or at least while the vehicle is in motion.

### BRIEF SUMMARY OF THE INVENTION

The present disclosure monitors a vehicle operator by tracking eye movements with a capacitive electrode positioned close to the operator's head. The electrode is capable of sensing electrical impulses travelling between the operator's brain and eyes in order to track movements of the operator's eyelids, such as the eyelids closing and subsequently opening. These movements can be monitored to determine how fast and how frequently the operator is blinking. Slow blinking may signal that the operator is falling asleep, frequent blinking may signal that the operator is conversing, and infrequent blinking may indicate that the operator is reading.

The present disclosure also improves upon the current technology by providing a casing into which the operator's mobile device may be placed. The casing includes a control circuit that monitors operation of the vehicle and determines whether it is safe for the operator to access the device. When it is not safe to access the device, the control circuit locks the device inside the casing, thereby preventing access. The determination may be informed by accelerometers with high sensitivity that can differentiate between an idle vehicle and a vehicle in motion based on the absence or presence of small vibrations from the vehicle's motor. These vibrations are normally felt when the vehicle is idle, but not when the vehicle is in motion.

One aspect of the disclosure is directed to a control unit for monitoring attentiveness of an operator. The control unit may include a wireless receiver configured to receive digital signals indicative of electrical activity induced by ion flow in the operator's head as a result of the operator blinking. The control circuit may be communicatively coupled to the wireless receiver and configured to identify a plurality of eye-closing events and a plurality of eye-opening events based on the received digital signal, and to determine a state of attentiveness of the operator based on the identified eye-closing and eye-opening events. In some examples, the control unit may be adapted to be mounted to a machine being operated by the operator.

The control unit may further include any one or combination of: one or more accelerometers configured to detect a magnitude of acceleration of the machine, such that a detected magnitude of acceleration from the one or more accelerometers may be used to determine whether the machine is in motion or idle; a housing adapted to house the control circuit, to encase a mobile device of the operator, or both; an alarm configured to notify the operator of a determination that the operator lacks attentiveness; a solenoid lock that may be adapted to lock the housing, such that the mobile device cannot be accessed by the operator when the housing is locked by the solenoid lock (e.g., when the mobile device is encased in the housing); a pressure sensor configured to sense a presence of the mobile device in the housing, such that the control circuit may be configured to receive an indication from the pressure sensor (e.g., that the mobile device is in the housing); and a bolt to lock the housing when the machine is determined to be in motion.

In some examples, the housing may further include a back casing, a hinged front panel, and a slot. The slot may be mounted to the front casing and adapted to receive a bolt mounted to the back casing. Actuating the bolt may cause the bolt to be inserted into the slot, thereby locking the hinged front panel to the back casing. The control unit may be configured to actuate the bolt when the mobile device is in the housing and the machine is in motion.

Another aspect of the disclosure is directed to a method for monitoring attention of a machine operator. The method may be executed by a processor and may involve: receiving data from a capacitive electrode positioned in proximity to the operator's head to detect an ion flow of electrical impulses between the driver's brain and eyelids; identifying a plurality of first events from the received data, each first event corresponding to the operator's eyes closing; for each identified first event, identifying a second event in the received data following the first event, the second event corresponding to the operator's eyes opening; and based on the identified first and second events in the received data, determining a lack of attentiveness of the operator.

In some examples, the method may further involve, for each pair of first and second events, comparing an interval between the first and second events to a maximum blink duration value, such that the interval exceeding the maximum blink duration value is indicative of a lack of attentiveness of the operator; and in response to determining a lack of attentiveness, at the processor, one of activating an alarm and logging a lack of attentiveness event in a datalog. In some examples, the ion flow being greater than or equal to a maximum threshold value may be indicative of one of a first event or a second event. In some examples, the magnitude of electrical activity being less than or equal to a minimum threshold value may be indicative of the other of the first event or second event.

In some examples, the method may further involve a calibration stage and a subsequent monitoring stage.

The calibration stage may involve: receiving data from the capacitive electrode for a preset calibration duration; for each pair of first and second events identified in the data received during the calibration duration, calculating an interval between the first and second events; and selecting the maximum interval from among the calculated intervals as the maximum blink duration value. For further example, the calibration stage may involve: collecting a set of received data over a preset calibration timespan; and calculating a baseline blink rate of the operator over the preset calibration timespan.

The monitoring stage may involve: receiving data from the capacitive electrode; identifying a first event from the data received during the monitoring stage, the first event corresponding to the operator's eyes closing; identifying a second event from the data received during the monitoring stage, the second event corresponding to the operator's eyes opening; and comparing an interval between the first and second events of the monitoring stage to the maximum blink duration value. For further example, the monitoring stage may involve: receiving data from the capacitive electrode for a preset duration; for each pair of first and second events identified in the data received during the duration, identifying a blink of the operator; calculating a blink rate of the operator over the duration based on the identified blinks; and comparing the calculated blink rate to each of a maximum blink rate value and minimum blink rate value, whereby the measured blink rate being greater than the maximum blink rate value or less than the minimum blink rate value may be indicative of a lack of attentiveness of the operator. The maximum blink rate value may derived from the baseline blink rate (e.g., set at a value at least 1.88 times greater than the baseline blink rate), and may indicate that the operator is engaged in a conversation. The minimum blink rate value may be derived from a baseline blink rate (e.g., set at a value at most 0.55 times the baseline blink rate) and may indicate that the operator is reading.

Yet another aspect of the disclosure is directed to a method for preventing distraction to a machine operator. The method may be executed by a processor, and may involve: upon receiving an indication of the machine being activated, initiating a calibration stage; during the calibration stage, collecting data from one or more accelerometers, each accelerometer indicating a magnitude of acceleration along a respective axis of the machine; for each accelerometer, based on the collected data during the calibration stage, calculating a threshold range of acceleration values; after the calibration stage, initiating a monitoring stage; and during the monitoring stage, collecting data from the one or more accelerometers, for each accelerometer, comparing the collected data from the accelerometer to the accelerator's calculated threshold range, and if the collected data is outside the corresponding threshold range, activating a lock to prevent distraction to the machine operator. The threshold range of acceleration values may correspond to a vibration of the machine when the machine is powered and stationary, whereby the machine being not powered or in motion may result in acceleration values falling outside of the threshold range.

In some examples, each or some subset of the one or more accelerometers may have a sensitivity of at least 10 mV/g. The accelerometers may measure acceleration along any combination of an up-down axis of the machine, a left-right axis of the machine, and a front-back axis of the machine.

In some examples, if a lock is activated, the method may further involve, determining whether data collected from each accelerator during the monitoring stage remains within the corresponding threshold range for a predetermined duration, and deactivating the lock based on the determination. If the machine is an automotive vehicle, activating the lock may involve locking a mobile device belonging to the operator in a casing, and deactivating the lock would permit the operator to retrieve the mobile device from the casing.

Yet another aspect of the disclosure is directed to a data acquisition unit for acquiring data from an operator. The data acquisition unit may include a pair of electrodes adapted to be positioned in proximity to the operator's head to detect an ion flow of electrical impulses between the operator's brain and eyelids associated with operator's eyes closing or opening, and electrical circuitry coupled to the pair of electrodes and configured to convert an electrical current received from the pair of electrodes into a digital signal. The data acquisition unit may further include one or both of: a housing adapted to encase the electrical circuitry; and a mounting mechanism positioned on an outer surface of the housing. The mounting mechanism may be adapted to be fastened to an article worn on the operator's head.

Each electrode may have a diameter of between about 10 mm and 30 mm and a thickness of between about 1.35 mm and 10.5 mm. Each electrode may contain a conductive copper filament. A blinking action of the operator may cause electrical current to be generated in the filament.

In some examples, the electrical circuitry may include some or all of: a wireless transmitter configured to wirelessly transmit the digital signal to a remote unit; a high pass filter configured to filter analog data received from the pair of electrodes; and a differential amplifier configured to remove noise generated by the operator's body. The amplifier may have a frequency between about 50 kHz and 60 kHz.

The present disclosure is also directed to a system for monitoring attentiveness of an operator having both a data acquisition unit and a control unit as described herein or in any of the above examples. In one instance, the data acquisition unit may be adapted to be worn by the operator, and may include a capacitive electrode configured to detect electrical activity induced by eye blinks of the operator. The system may include a converter circuit configured to process the detected electrical activity into a digital signal, and a wireless transmitter configured to wirelessly transmit the digital signal. The system may also include a control unit positioned remote from the data acquisition unit. The control unit may include a wireless receiver configured to receive the digital signal from the wireless transmitter, and a control circuit communicatively coupled to the wireless receiver to receive the digital signal. The control circuit may be configured to identify a plurality of eye-closing events and a plurality of eye-opening events from the digital signal, and to determine a state of attentiveness of the operator based on the identified eye-closing and eye-opening events.

An additional aspect of the disclosure is directed to a control circuit having a processor and memory, the memory storing data and instructions necessary for carrying out any of the methods described herein.

These and further aspects of the disclosure are described in greater detail in reference to the following drawings.

### BRIEF DESCRIPTION OF THE DRAWINGS

FIG. 1 is an illustration of an operator operating a machine in accordance with an aspect of the disclosure.

FIG. 2A is an illustration of a data acquisition unit in accordance with an aspect of the disclosure.

FIG. 2B is a side view of the data acquisition unit of FIG. 2A.

FIG. 3 is a circuit diagram of the data acquisition circuit of FIGS. 2A and 2B.

FIG. 4 is an illustration of a control unit in accordance with an aspect of the disclosure.

FIG. 5 is a functional block diagram of the control unit of FIG. 4.

FIG. 6 is a flow diagram of a calibration routine in accordance with an aspect of the disclosure.

FIG. 7 is a flow diagram of an operator attentiveness monitoring routine in accordance with an aspect of the disclosure.

FIG. 8 is a flow diagram showing steps of the routine of FIG. 7 in greater detail.

FIG. 9 is a flow diagram of an operational activity monitoring routine in accordance with an aspect of the disclosure.

### DETAILED DESCRIPTION

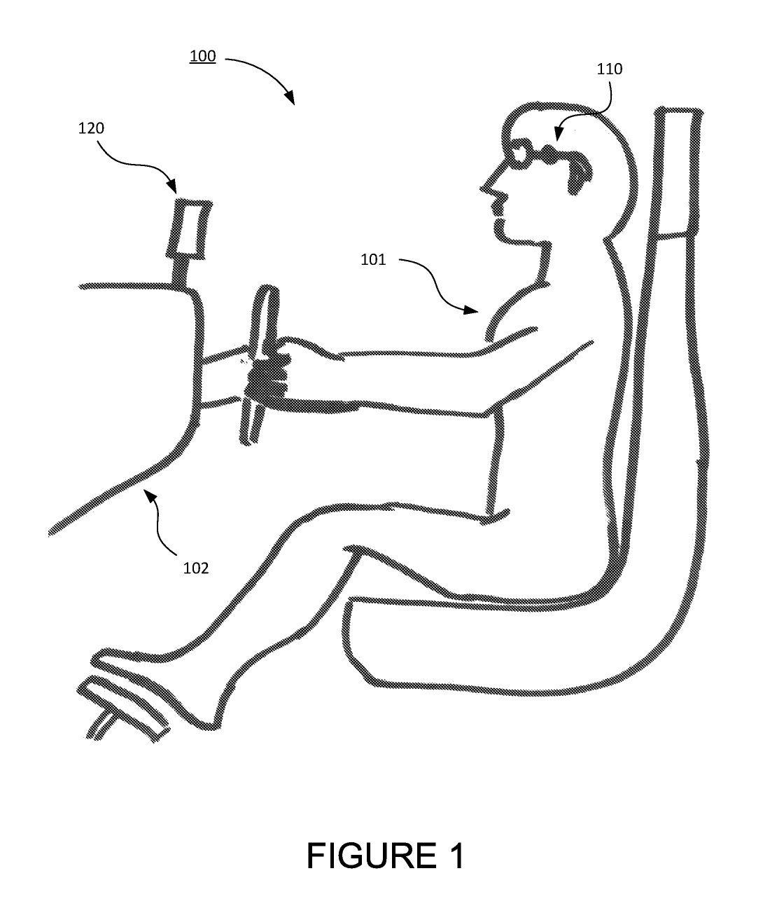

FIG. 1 illustrates an example system 100 in which an operator or driver 101 operates a machine, such as a vehicle 102. A data acquisition unit 110 is positioned touching or in close proximity to the operator's head. In the example of FIG. 1, the data acquisition unit 110 is mounted to a pair of eyeglasses of the driver 101. Additionally, a control unit 120 is positioned in the vehicle 102. In the example of FIG. 1, the control unit 120 is mounted to the dashboard of the vehicle 102. The data acquisition unit 110 is wirelessly connected to the control unit 102 to permit for wireless communication of data from the data acquisition unit 110 to the control unit 120.

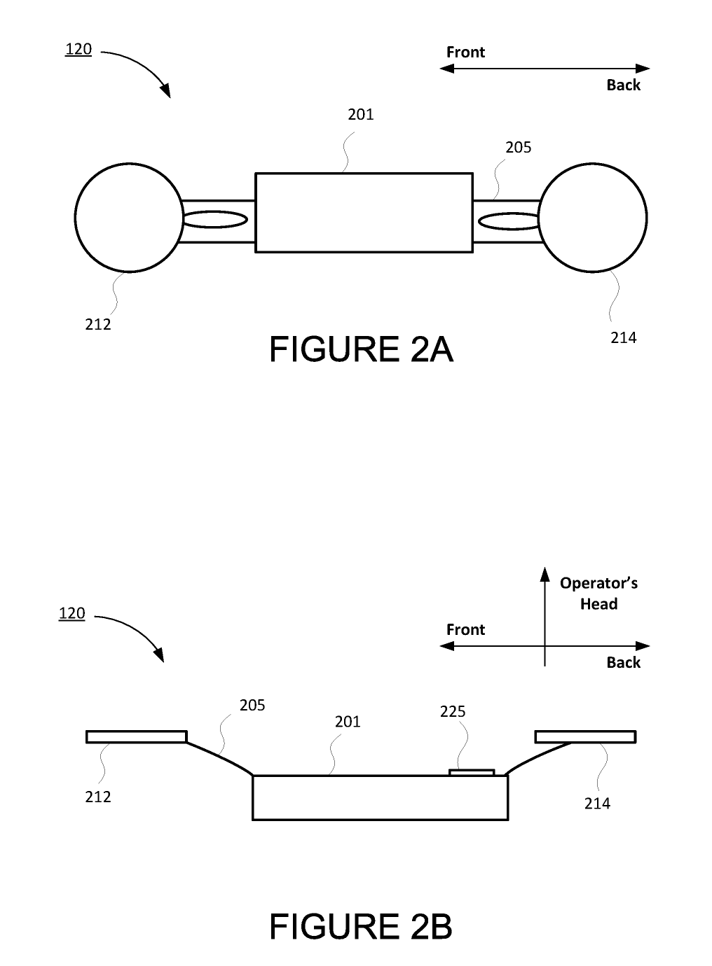

FIGS. 2A and 2B illustrate an example structure of the data acquisition unit 110. The data acquisition unit 110 includes a body 201 positioned between each of an active electrode 212 and a ground electrode 214. The body 201 is connected each electrode by a curved extension member 205 (curvature shown in FIG. 2B).

The body 201 is a box-shaped housing, having a length of between about 80 mm and 32 mm, and preferably about 60.65 mm, a width of between about 20 mm and 11.5 mm, and preferably about 15.75 mm, and a thickness of between about 10 mm and 3.2 mm, and preferably about 8.08 mm The body 201 may contain a power supply for powering the data acquisition unit 110, such as a lithium ion battery. The body may also include a connection port, such as a universal serial bus (USB) port for plugging in the unit in order to recharge the battery.

Also, as shown in FIG. 2B, an outer surface of the body may include a mounting mechanism 225 for mounting the data acquisition unit 110 to the driver 101, such as a clasp, hook, latch, sleeve, adhesive, or any other fastening means known in the art. The mounting mechanism may be configured to permit the data acquisition unit to be mounted other articles worn on the driver's head, including but not limited to sunglasses, protective eyewear, headphones and headwear, including hats and headbands.

Each electrode is a disc-shaped housing having a diameter of between about 30 mm and 10 mm, and preferably about 24.26 mm, and a thickness of between about 10.5 mm and 1.35 mm, and preferably about 5.25 mm Each electrode contains a conductor that is sensitive to electrical activity induced by eye blinks of the driver 101.

For instance, each electrode may be a capacitive electrode or plate that is sensitive to neuronal ion channel dynamics taking place in the operator's brain. The electrode is placed in sufficient proximity to the skull of the operator in order for the skull and electrode to effectively behave like a capacitor. The electrode may further include a conductive material, such as copper filament, to attract displacement currents. In this regard, the blinking action of the driver 101 can result in an electric flux due to the movement of electrons between the driver's brain and eye, and this electric flux can generate displacement currents flowing from the driver's skull to the conductive material of the electrode. The displacement current, by nature, is an AC current that may flow from the electrode to the data acquisition unit circuitry to be converted into an electrical signal.

The blinking action can be thought of as a pair of two consecutive actions: an eye closing and a subsequent eye opening (technically, re-opening). Each of these actions is controlled by electrical impulses delivered from the operator's brain to the eyelids, whereby the ion flow for closing the eyelids is in reverse compared to the ion flow for opening the eyelids. In this respect, the working electrode 212 can detect the ion flow of the electrical impulses associated with the eyes of the driver closing and then opening. Comparing the detected ion flow to the neutral ion flow detected by the neutral electrode can be used to classify eye-closing events (ion flow causing electrical current in the filament to flow in one direction) from eye-opening events (ion flow causing the electrical current to flow in the other direction).

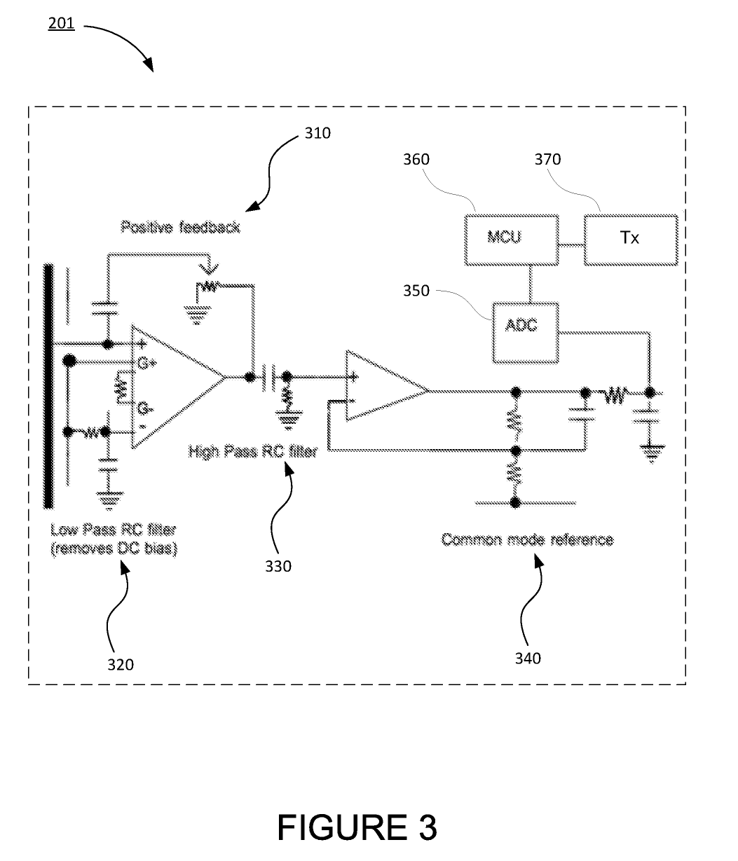

Electrical current from the electrodes 212, 214 is transmitted by wire through the extension members 205 to the body 201. The body 201 further contains circuitry for processing the electrical current and transmitting the processed information as a data signal. FIG. 3 shows an example circuit diagram of the circuitry that may be housed in the body 201. In the example of FIG. 3, the analog electrical signal from the positive electrode 212 is provided as the non-inverting input to a positive feedback amplifier 310, and the ground electrode may be connected to ground. The analog signal is further fed from the non-inventing input guard to the inverting input of the amplifier 310 via a low pass filter 320 to negate any direct current bias from the signal. The output of the amplifier 310 is then processed through a high pass filter 330. The high pass filter 330 passes the abrupt electrical fluctuations caused by the eye-closing and eye-opening events, while blocking electrical noise at lower frequencies. The cutoff frequency of the high pass filter may be set between 3 Hz and 0.25 Hz, and preferably to about 1 Hz. The high pass filter 330 output is then provided as the non-inverted input of a differential amplifier 340. The inverted input of the amplifier 340 is a common mode signal, thereby canceling out noise from the driver's body picked up by both the active electrode 212 and the ground electrode 214. The differential amplifier 340 is useful for removing 50-60 Hz noise that results from the body's antenna-like behavior when probed by an electrode.

The differential amplifier 340 output is provided to an analog-digital-converter (ADC) 350 for sampling. The digitized signal is provided to a microcontroller 360 through a serial peripheral interface (SPI), and from the microcontroller is passed to a transmitter 370, such as a Bluetooth module, for transmission to the control unit 120 remote from the data acquisition unit 110.

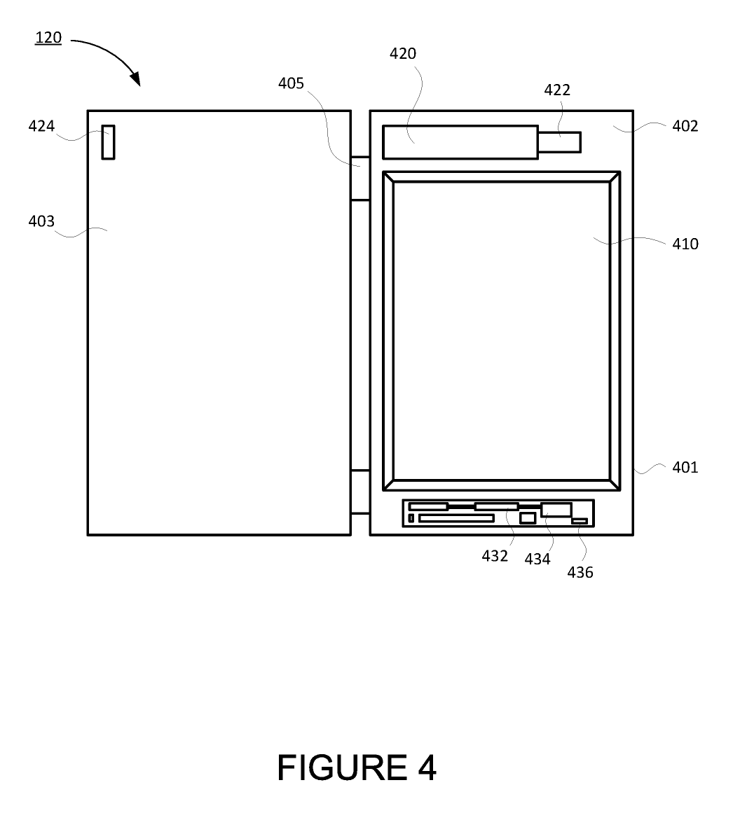

FIG. 4 illustrates an example structure of a control unit 120. The control unit 120 includes a housing or casing 401 including each of a back panel 402 and front panel 403 connected by one or more hinges 405. The back panel includes a recess or cavity to accommodate a mobile device of the driver. In the example of FIG. 4, the cavity has a rectangular shape and is slightly taller, wider and deeper than a typical smartphone. The front panel 403 is adapted to pivot at the hinges 405 in order to removably cover the recess 410.

The control unit 120 is adapted to encase and lock the driver's mobile device. In the example of FIG. 4, the locking mechanism is positioned above the recess 420, and includes a dead bolt body 420, and slot 424. The dead bolt body 420 is mounted to an inner surface of the back panel 402. The dead bolt body 420 includes a dead bolt 422 than can be actuated by a solenoid, which in turn may be controlled by an electrical current provided according to instructions from the control unit 120. The slot is mounted to an inner surface of the front panel, such that when the front panel 403 is folded over the back panel 402 to cover the recess 410, the slot 424 is aligned with the dead bolt 422. Upon actuation, the solenoid causes the dead bolt 422 extends out of the dead bolt body 420, and when aligned with the slot 424, slides into the slot 424, thereby locking the housing 401.

The control unit housing 401 is also adapted to house electronic components. In the example of FIG. 4, the components are positioned underneath the recess 410, and include a wireless receiver 432, a control circuit 434, an accelerometer 436 and an alarm 438. The wireless receiver 432 is adapted to receive the data signals transmitted by the wireless transmitter of a data acquisition unit. For example, if the transmitter is a Bluetooth transmitter, then the receiver 432 is a Bluetooth receiver. The receiver may also be configured to receive wireless signals from other sources, such as a wireless signal from the vehicle indicating that the vehicle is on.

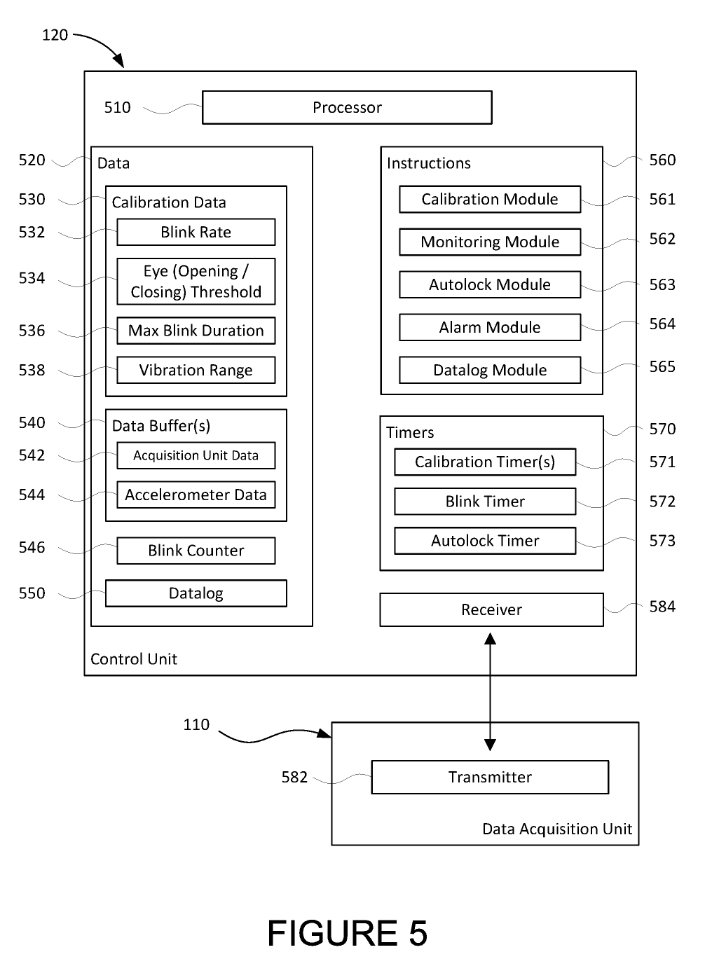

The control circuit 434 is configured to further process the data signals received from the data acquisition unit and accelerometer 436, and to manage the routines of the present disclosure described herein. The components of the control circuit are described in greater detail in connection with FIG. 5.

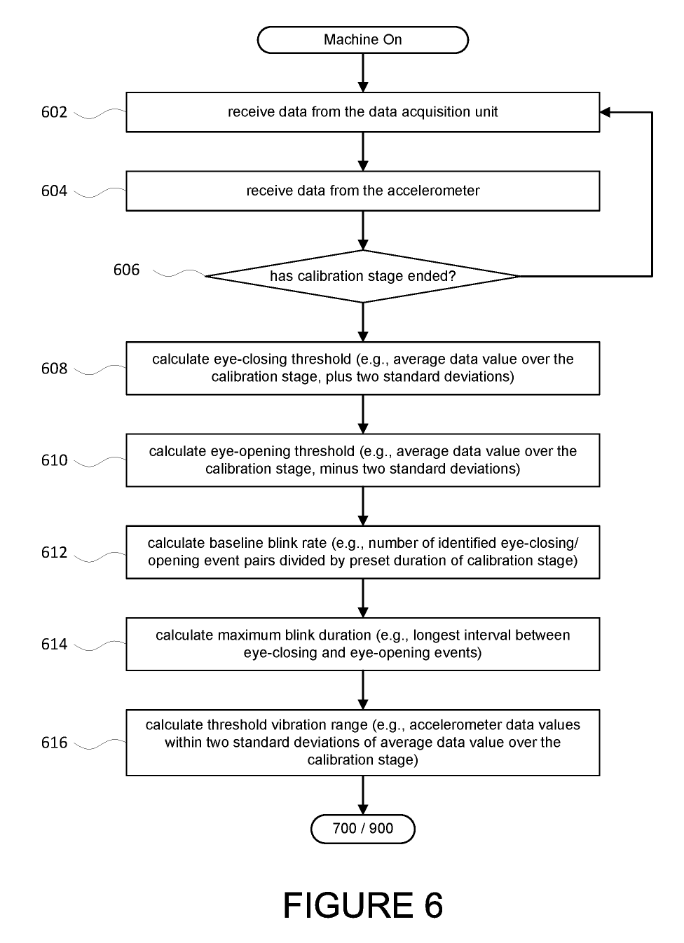

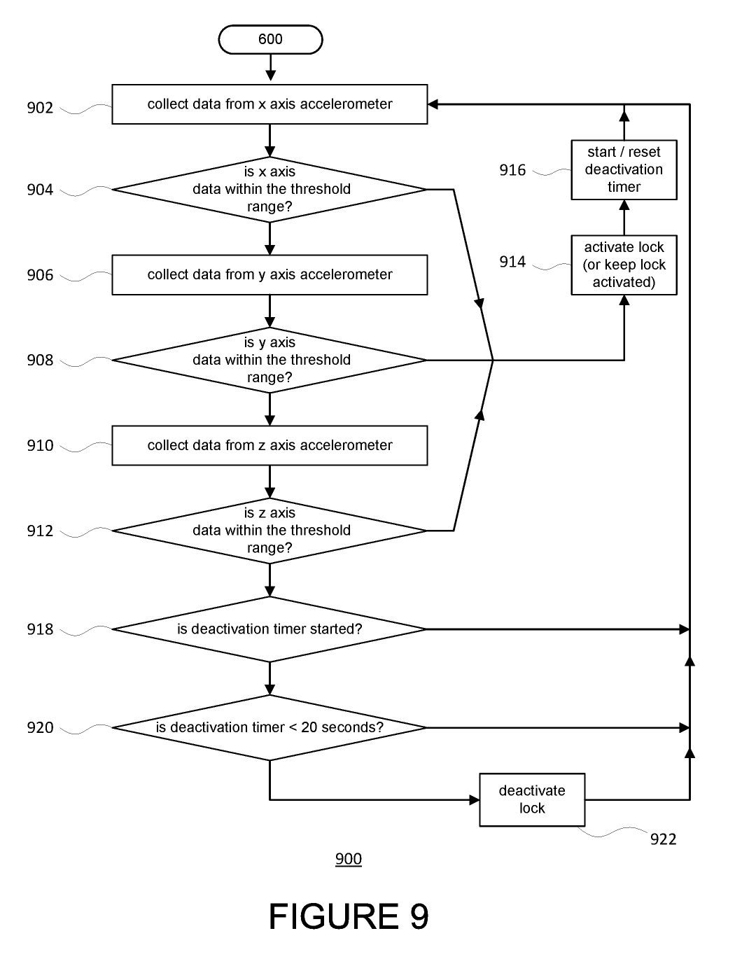

The accelerometer 436 is provided to sense motion of the vehicle, such as differentiating between when the vehicle is idle and when it is in motion. When the vehicle is idle, the vehicle motor may cause the vehicle to vibrate more than when it is in motion. These vibrations may be picked up by a sufficiently sensitive accelerometer having a sufficiently long sampling rate. The absence of the vibration motions can then be used to determine when the vehicle is in motion instead of idle. The accelerometer may have a sensitivity of between about 10 mV/g and 15,000 mV/g, and preferably at least about 500 mV/g. The accelerometer may also have a sampling rate (time constant) of between about 0.25 seconds to about 10 seconds, and preferably at least about 1 seconds. Routines involving the accelerometer 436 are described in greater detail in connection with each of FIGS. 6 and 9.

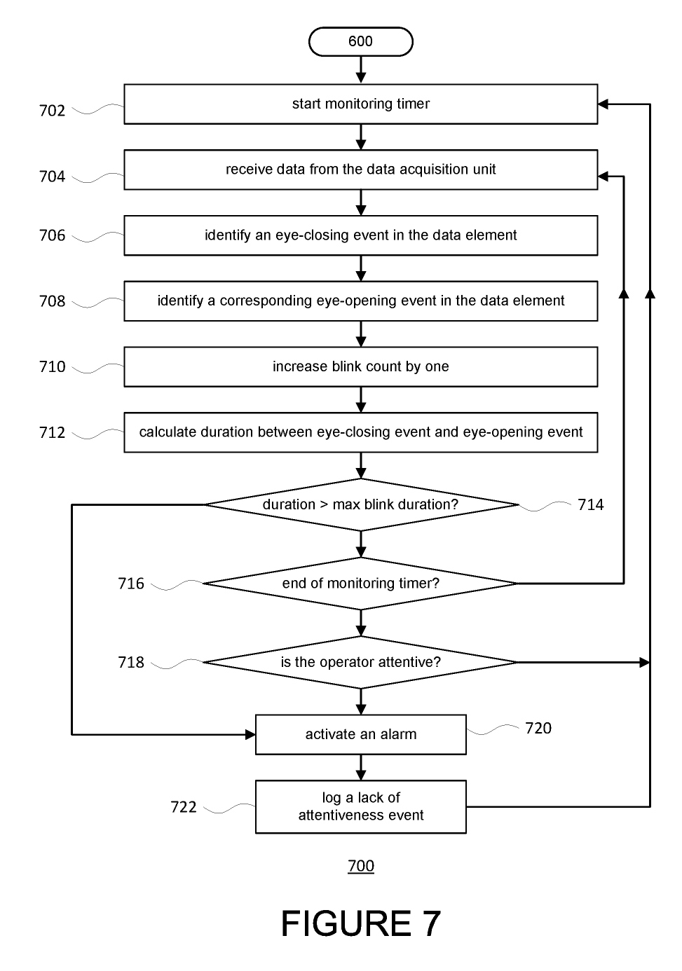

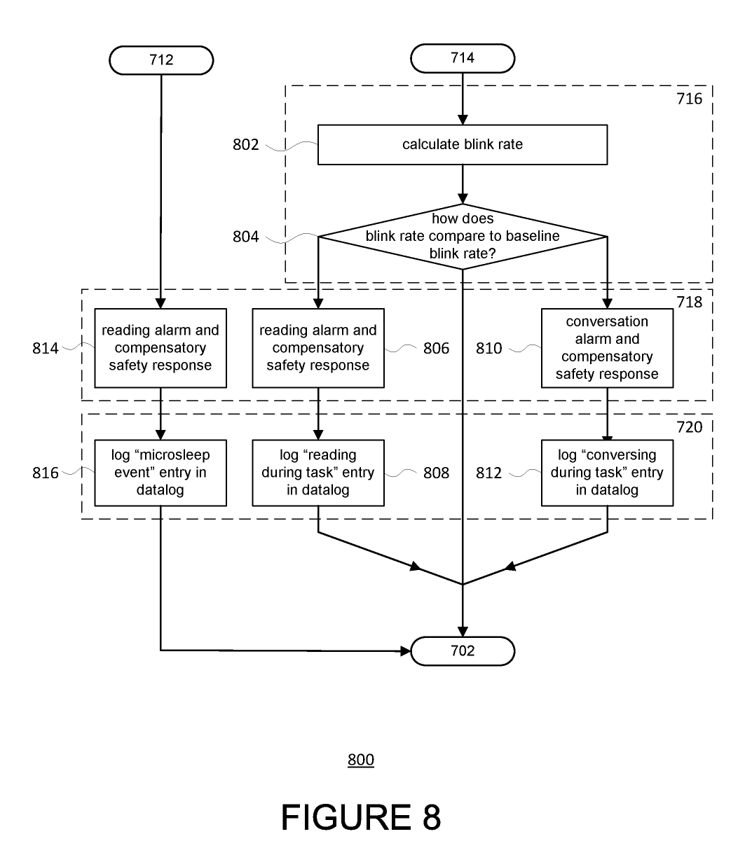

The alarm 438 is provided to alert the driver when the control unit 120 determines that driver is distracted or lacks attentiveness (e.g., reading, conversing, falling asleep, etc.) while operating the vehicle. Routines involving the alarm are described in greater detail in connection with each of FIGS. 7 and 8.

The control unit 120 may also include a pressure sensor (not shown) in order to detect when a mobile device is placed in the control unit 120. In one instance, the pressure sensor may be positioned inside the recess 410, such that placing the mobile device in the recess increases the pressure applied to the sensor. The sensor may be wired to the control circuit 434 to provide an indication of whether the recess 410 is occupied or vacant. In turn, the control circuit 434 may be programmed to lock the control unit 120 when the recess 410 is occupied by the mobile device.

FIG. 5 is a functional block diagram of the system 100, including the data acquisition unit 110 and the control unit 120. As seen in FIG. 5, the control unit 120 includes a processor 510, memory storing each of data 520 and instructions 560, and one or more timers 570. The memory may be a component configured to store a large quantity of data, for example, any one or collection of ROM, RAM, hard-drives, solid-state drives, removable drives, multi-leveled cache, registers, buffers, etc. In addition, the memory may be configured so that it can be accessed by the processor 510 in order to carry out the instructions 560.

The data 520 may include several values, thresholds and ranges that are useful for carrying out the instructions. Some of the data may be preset in the memory before beginning to carry out the instructions, whereas other data is collected, analyzed and determined as part of the routines described herein. Additionally, some of the data stored in the memory may be temporarily stored and then erased after it has been analyzed and processed, whereas other data in the memory may be permanently or indefinitely stored.

The data 520 includes several values collected during an initial calibration stage, referred to as calibration data 530. During the initial calibration stage, it is assumed that the driver is alert and attentive (e.g., not having a conversation, not falling asleep, not reading) and that the vehicle is on but not moving. Thus, the data collected during this initial calibration stage is indicative of the driver's standard behavior when attentive, and the vehicle's standard behavior when stationary. Stated another way, the calibration data 530 can be used as a baseline for monitoring the driver's and vehicle's behavior at a later time, such as during a subsequent monitoring stage. The particular baseline values collected during calibration may include a baseline blink rate 532, baseline eye opening and eye closing thresholds 534, a maximum blink duration 536 and a baseline vibration range 538. Methods of deriving these values are discussed in greater detail in connection with FIG. 6.

The data 520 in the memory also includes one or more buffers 540 capable of temporarily storing a stream of incoming data. The buffered data may include data received from the data acquisition unit 542 and data received from the accelerometer 544. The buffers may be FIFO buffers, permitting the control unit to process and analyze the data in the order it is received. The buffers may be used both during the calibration stage (e.g., to derive the baseline and threshold values) as well as during the monitoring stage (e.g., to derive current values for comparison with the baseline values and thresholds)

Processing and analyzing the buffered data may involve determining new values based on the analysis. The calibration data 530 are examples of new values derived from analyzing buffered data. Other values may be derived during the monitoring stage. These values are referred to as monitoring data.

The data 520 may further include one or more counters for tracking a tallied value. One example is a blink counter 546. The blink counter tracks the total number of times the driver blinks over a given duration. Counting the total number of blinks is useful during the calibration stage to derive a baseline blink rate 532. Counting the total number of blinks is also useful during the monitoring stage to derive a then-current blink rate that may be compared to the baseline blink rate 532.

Also included in the memory is a datalog 550 of recorded events, such as the vehicle being activated or deactivated, the driver's mobile device being inserted into or removed from the control unit, or the driver becoming inattentive or distracted. Each datalog entry may include a timestamp indicating the time of the event. The datalog may be stored in non-volatile memory to ensure storage of the logged events for analysis at a later time. For example, the data may be provided to an insurer of the driver in order to exclude whether or not the driver is operating the vehicle safely.

The instructions 560 in the memory include one or more modules for carrying out the routines described herein. In the example of FIG. 5, the instructions 560 include a calibration module 561 for executing a calibration stage, a monitoring module 562 for monitoring the driver's state of attentiveness and alertness during the monitoring stage, an autolock module 563 for monitoring whether the vehicle is in operation and controlling whether the driver's mobile device is locked in the control unit during the monitoring stage, an alarm module 564 for alerting the driver if it is determined that the driver is not attentive or alert, and a datalog module 565 for recording events to the datalog 550. The modules shown in FIG. 5 are merely examples and not intended to represent a complete or detailed list of the operations executed by the control unit.

The control unit 120 further includes one or more timers for tracking the durations of various stages, routines or events. One such timer may be a calibration timer 571, used to track the duration of the calibration stage and determine when the calibration stage is complete. In one example, the calibration stage may last one minute, meaning that the vehicle should remain on but stationary before the driver begins driving the vehicle. The driver should also stay alert and undistracted (e.g., not conversing with another passenger or over a phone, not closing their eyes, not reading) for the duration of the calibration stage. Separate timers may be used for each of calibrating the vehicle's and the driver's behaviors, and the separate timers may optionally start at different times, be set for different durations, or both.

A blink timer 572 can be used to track the duration of a driver's blink. The duration of a blink may be thought of as the time from when the driver closes their eyes until the time when the driver reopens their eyes. The blink timer 572 is useful during the calibration stage to calculate the baseline blink rate 532, as well as during the monitoring stage to calculate a current blink rate for comparison with the baseline blink rate 532.

In the case of the control unit locking the driver's mobile device, an autolock timer 573 can be used to track how much time has passed since the most recent determination to lock the device or keep the device locked. Until the autolock timer reaches a preset duration, the device remains locked in the control unit 110. In one example, the autolock timer 573 can be set to about 20 seconds, meaning that the control unit 120 should determine that the vehicle has been stationary for at least 20 seconds before the mobile device is unlocked.

Also shown in FIG. 5 are the transmitter 582 of the data acquisition unit 110 and the receiver 584 of the control unit 120. The transmitter and receiver are capable of transferring the data acquired at the data acquisition unit 110 over to the control unit 120 for further processing. Not shown but also included in the control unit 120 is the accelerometer (436 in FIG. 4).

In the example of FIGS. 3, 4 and 5, the system 100 is shown as supporting only one-way communication from the data acquisition unit 110 to the control unit 120. However, those skilled in the art will readily appreciate that other example systems could be configured to support two-way communication. For instance, a control unit could be configured to send control signals to a data acquisition unit in order to turn the data acquisition unit on or off depending on various factors (e.g., not receiving data from the data acquisition unit for a period of time, detecting that the data acquisition unit is mounted to the control unit, etc.). Such two-way communication features could be beneficial, such as to reduce drain on the data acquisition unit battery.

Also, in the example of FIGS. 4 and 5, the control circuitry of the control unit 120 is included in the housing of the control unit. However, those skilled in the art will readily appreciate that the control circuitry can be fully or partially stored apart from the control unit without departing the spirit of the present disclosure. In such an instance, the control unit could include a transceiver for transmitting data wirelessly to a remotely stored control circuit, and receiving control signals from the remote control circuit. The control signals could activate alarms, control locking the driver's mobile device in the control unit, or perform other functions of the control unit.

The systems and devices described above may be operated using the example routines described herein. It should be understood that the operations of the following routines do not have to be performed in the precise order described below. Rather, various operations can be handled in a different order, or simultaneously. Moreover, operations may be added or omitted.

FIG. 6 is a flow diagram showing an example calibration routine 600 executed by the control unit processor upon the vehicle being turned on and the calibration timer 571 being initiated. At block 602, the processor receives data from the data acquisition unit. The data may indicate a magnitude of electrical activity generated at the capacitive electrode of the data acquisition unit. More particularly, the data may indicate a magnitude of ion flow of electrical impulses between the driver's brain and eyelids. At block 604, the processor receives data from the accelerometer. The accelerometer data may indicate a magnitude of acceleration along a respective axis of the vehicle. At block 606, the processor checks whether the calibration duration has elapsed. If the calibration duration has not elapsed, the operations of blocks 602 and 604 are repeated. Thus, data from the data acquisition unit and the accelerometer are collected for the entire duration of the calibration stage until the calibration duration elapses (e.g., after one minute).

After the calibration elapses, operations proceed with calculating calibration values based on the calibration data. The calibration values can then be stored in the control unit memory for use during the subsequent monitoring stage.

At block 608, an eye-closing threshold is calculated. In one example, the eye-closing threshold may be calculated by calculating an average and standard deviation of the magnitude of ion flow of electrical impulses between the driver's brain and eyelids, and then setting the eye-closing threshold at a value equal to two standard deviations greater than the calculated average. In this respect, any time the detected ion flow is more than two standard deviations above the average ion flow, such an occurrence may be classified as an eye-closing event.

At block 610, an eye-opening threshold is calculated. The eye-opening threshold may be calculated in a manner similar to the eye-closing threshold. In other words, the eye-opening threshold may be set at a value equal to two standard deviations less than the calculated average. In this respect, any time the detected ion flow is more than two standard deviations below the average ion flow, such an occurrence may be classified as an eye-opening event.

At block 612, a baseline blink rate is calculated. The baseline blink rate is the blink rate of the driver over the duration of the calibration stage. This value may be calculated by pairing each of the classified eye-opening events with their corresponding subsequent eye-closing events to create a blink event, tallying the total number of blink events during the calibration duration, and dividing by the calibration duration. For example, if the calibration stage takes one minute, and the driver blinks 12 times during that time, then the baseline blink rate is 12 blinks per minute.

At block 614, a maximum blink duration is calculated. The maximum blink duration may be the longest duration of any blink during the calibration stage. Blink duration can be measured using the blink timer 572. The timer is started upon detection of an eye-closing event, and stopped upon detection of a subsequent eye-opening event the elapsed time is measured and recorded. As each blink event is iteratively recorded, the elapsed time of the blink event may be compared to that of the previous blink event, and the longer time of the two blink events may be stored as the maximum blink duration. Thus, upon completion of the calibration stage, the longest time of the blink events is the maximum blink duration.

At block 616, a vibration range is calculated. The vibration range may be the typical acceleration of the vehicle when it is on but stationary, meaning that the vehicle is vibrating. The vibration range can be collected in a manner similar to the eye-opening and eye-closing thresholds. The accelerometer data collected over the duration of the calibration stage can be averaged, and standard deviation can be determined. The vibration range can then be set as a range of values within a certain number of standard deviations of the average (e.g., within two standard deviations).

Since the typical vibrations of a stationary vehicle may differ depending on the axis measured, vibrations along multiple axes of the vehicle can be collected and calibrated. For instance, the control unit can include three accelerometers: one for measuring vibrations along the left-right axis of the vehicle; one for measuring vibrations along the front-back axis; and one for measuring vibrations along the vertical axis. Independent vibration ranges may be computed for each accelerometer.

After the calibration values have been collected and stored, operations may proceed to the monitoring stage routines described in connection with FIGS. 7-9.

FIG. 7 is a flow diagram showing an example monitoring routine 700 executed by the control unit processor 510 upon completion of the calibration routine 600. At block 702 the blink timer 572 is started, which may be the same timer as used in the calibration stage.

At block 704, the processor receives data from the data acquisition unit. As mentioned above, the received data may indicate a magnitude of ion flow of electrical impulses between the driver's brain and eyelids.

At block 706, the processor identifies an eye-closing event in the received data. For example, the processor may compare each piece of received data to the eye-closing threshold value. When a piece of data is found to exceed the eye-closing threshold value, the processor may interpret the data at indicative of an eye-closing event.

Upon detection of the eye-closing event, operations may then continue at block 708 with the processor identifying a corresponding eye-opening event in the received data. For example, the processor may compare each piece of received data to the eye-opening threshold value. When a piece of data is found to be less than the eye-opening threshold value, the processor may interpret the data at indicative of an eye-opening event.

Since every blink involves the driver's eyes both closing and then subsequently opening, the processor may be programmed to search for eye-opening events only after identifying an eye-closing event. In other words, if the processor receives data corresponding to consecutive eye-closing events, the processor may be programmed to identify only one of the events—either the former event or the latter event—as an eye-closing event and discard the other event it is not paired with a corresponding and subsequent eye opening event.

Once both an eye-closing event and subsequent eye-opening event have been identified, the pair of events may be identified as a blink. At block 710, the blink counter 546 may be increased by one, thereby tallying the identified blink. Additionally, at block 712, the duration of the blink may be calculated by taking the difference in time or interval between the eye-closing event and the eye-opening event. Taking the difference in time may be achieved by analyzing timestamps of the received data. Alternatively, if the processor generates its own timestamps for the eye-closing and eye-opening events, those timestamps may be compared with one another.

At block 714, the duration of the blink may be compared to the maximum blink duration value 536 collected at the calibration stage. In essence, this comparison determines whether the duration of the most recently identified blink was longer than any blink detected when the driver was known to be alert during the calibration stage. The blink duration exceeding the maximum blink duration value 536 may indicate that the driver's eyes are beginning to droop and that the driver is beginning to fall asleep while driving, and thus trigger an alarm at block 720 and result in a lack-of-attentiveness event being logged in the datalog 548 at block 722.

In the event that the driver's blink does not exceed the maximum blink duration value, operations may proceed at block 716 with checking of the blink timer 572. If the blink timer has not yet reached its preset goal (e.g., one minute), then operations revert to block 704 for the processor to continue receiving and analyzing data from the data acquisition unit. The operations of blocks 704-716 repeat themselves until the blink timer 572 reaches its preset goal, unless the driver is found to have begun falling asleep while driving and triggers an alarm.

When the blink timer 572 reaches the preset goal, operations continue at block 718 with the processor determining whether the driver is attentive or not based on the collected and processed data. If the driver is found to be attentive, the monitoring routine 700 begins from the start with operations reverting to block 702. The blink timer 572 is reset to allow for the next group of collected data to be analyzed. Otherwise, if the driver is found to not be attentive, then an alarm may be activated at block 720 and a lack-of-attentiveness event may be logged in the datalog 548 at block 722.

FIG. 8 is a flow diagram showing an example of how the processor determines whether a driver is attentive and how the processor responds. Blocks 718, 720 and 722 of routine 700 are shown in greater detail in FIG. 8.

Operations begin at block 802 with the processor calculating a blink rate of the driver for the data analyzed for the blink timer's duration. The blink rate may be the tally stored in the blink counter 546 divided by the duration of the blink timer 572. Then, at block 804, the processor compares the calculated blink rate against the baseline blink rate 532 determined during the calibration stage. This comparison can indicate whether the driver has been blinking faster than normal, slower than normal, or a relatively normal amount. “Normal” blinking is defined by the driver's blink rate during the calibration stage when it is assumed that the driver is attentive and not distracted. “Abnormal” blink rates may trigger alarms (block 720), datalog entries (block 722) or both, whereas a “normal” blink rate does not trigger an alarm or datalog entry.

If the calculated blink rate is determined to be less than the baseline blink rate by a threshold amount, such as 45% less than the baseline blink rate (i.e., 0.55 times the baseline blink rate), then it may be assumed that the driver was reading (e.g., a text message on a mobile device, a printed document present in the vehicle, etc.) during the monitoring stage. Operations then continue at block 806 in which the processor activates a reading alarm and compensatory safety response, by which the driver is alerted to the reading behavior and encouraged to stop reading. For example, the alarm may include a light (e.g., LED included on an outer surface of the control unit housing) or audio cue (e.g., generated by a speaker housing inside the control unit). At block 808, a “reading during task” entry is entered in the datalog 548.

If the calculated blink rate is determined to be greater than the baseline blink rate by a threshold amount, such as 88% greater than the baseline blink rate (i.e., 1.88 times the baseline blink rate), then it may be assumed that the driver was having a conversation (e.g., a phone call over a mobile device, a conversation with a passenger in the vehicle, etc.) during the monitoring stage. Operations then continue at block 810 in which the processor activates a conversation alarm and compensatory safety response, by which the driver is alerted to the conversation behavior and encouraged to stop conversing. For example, the alarm may include a light or audio cue that may be the same or different from the reading alarm. At block 812, a “conversing during task” entry is entered in the datalog 548 for storage.

If the blink rate of the driver is neither too far above nor too far below the baseline blink rate 532, then operations may continue at block 702 with resetting the blink timer 572, as described above in connection with FIG. 7.

FIG. 8 also shows that a different alarm and datalog entry may be used in the case of a driver who begins falling asleep. If a blink of the driver exceeds the maximum blink duration 536, operations may continue at block 814 with activation of a sleep alarm and compensatory safety response. The response may be designed not only to notify the driver of the sleep event, but may also be designed to wake the driver up. For example, if the alarm is an aural alarm, it may be louder than the reading and conversation alarms. At block 816, a “microsleep event” entry is logged in the datalog 548 for storage.

FIG. 9 is a flow diagram showing another example monitoring routine 900 executed by the control unit processor 510 upon completion of the calibration routine 600. The example of FIG. 9 describes a processor collecting and analyzing data from three accelerometers, but the same principles apply to any number of accelerometers (one or more).

At block 902 the processor collects data from an x-axis accelerometer that measures changes in the vehicle's motion in a forward-back direction. As noted above, the accelerometer may be configured with a sensitivity and sampling rate that are suitable for detecting the small changes in acceleration of an idle vehicle as its motor causes it to vibrate.

At block 904, the accelerometer data is compared to the threshold vibration range 538 of the x-axis accelerometer obtained during the calibration stage. If the accelerometer data falls within that threshold vibration range 538, it may be determined that the vehicle is vibrating in a manner similar to when the vehicle was idle during the calibration stage, thereby indicating that the vehicle is also currently idle. In this case, operations may proceed with block 906.

However, if the accelerometer data does not fall within the threshold vibration range 538, it may be determined that the vehicle is not vibrating in the same manner as when it was idle. In one case, the magnitude of the accelerometer data may fall below the threshold range, which may indicate that the vehicle is in motion at a relatively steady pace. In another case, the magnitude of the accelerometer data may be greater than the threshold range, which may indicate that the vehicle is speeding up or braking. In either of those cases, it may be determined that the vehicle is not idle, and operations may proceed with block 914, discussed further below.

Returning to block 906, the operations of blocks 906 and 908 are comparable to those of blocks 902 and 904 described above. The only difference is that the accelerometer data received and analyzed in these operations comes from a y-axis accelerometer that measures changes in the vehicle's motion in a side-to-side direction, and the threshold vibration range to which the y-axis accelerometer data is compared is also determined from y-axis accelerometer data during the calibration stage. If the collected data falls with the threshold range, then it may be determined that the vehicle is idle. Otherwise, if the data falls outside of the range, this may be determined to be indication that the vehicle is in motion. Collected data below the threshold may indicate steady motion of the vehicle, which can suppress the vibrations that are normally felt only when the vehicle is stationary. Collected data above the threshold may indicate the moving vehicle making a turn. Similar to blocks 902 and 904, operations continue at block 914 if the collected data falls outside the threshold range. Otherwise, if the data falls within the threshold range, operations continue at block 910.

The operations of blocks 910 and 912 are also comparable to those of blocks 902 and 904. The only difference is that the accelerometer data received and analyzed in these operations comes from a z-axis accelerometer that measures changes in the vehicle's motion in a vertical direction, and the threshold vibration range to which the z-axis accelerometer data is compared is also determined from z-axis accelerometer data during the calibration stage. If the collected data falls with the threshold range, then it may be determined that the vehicle is idle. Otherwise, if the data falls outside of the range, this may be determined to be indication that the vehicle is in motion. Collected data below the threshold may indicate steady motion of the vehicle, which can suppress the vibrations that are normally felt only when the vehicle is stationary. Collected data above the threshold may indicate the moving vehicle driving over a hill, pothole or other topologically significant road feature. Similar to blocks 902 and 904, operations continue at block 914 if the collected data falls outside the threshold range. Otherwise, if the data falls within the threshold range, operations continue at block 918, addressed further below.

Returning to block 914, in the case that any of the data collected from the accelerometers falls outside of the respective threshold ranges, the processor activates a lock to on the control unit. As discussed in connection with FIG. 4 above, the lock may be a bolt configured to lock the driver's mobile device inside the casing of the control unit. In this regard, the control unit processor detects when the vehicle is idle or moving and activates the lock on which the accelerometer data indicates that the vehicle is moving, thereby preventing the driver from the accessing the mobile device only while the vehicle is moving.

At block 916, an autolock timer 573 is initiated to keep track of when the vehicle was first detected going into motion. Since the stream of accelerometer data can from time to time fall within the threshold range even when the vehicle is moving, it is desirable to not immediately unlock the mobile device until the accelerometer data remains within the threshold range for a sufficient amount of time to ensure confidence that the vehicle is in fact idle. In one example, this amount of time may be about 20 seconds, although shorter time spans can also be used (e.g., 2 seconds, 5 seconds, 10 seconds, etc.). Alternatively, longer time spans may be used, such as to avoid a mobile device being freed while a car sits at a traffic light. Once the autolock timer 573 has been initiated, operations may revert to the beginning of the routine 900 with the next data from the x-axis, y-axis and z-axis accelerometers being collected and analyzed at blocks 902-912.

If the processor identifies accelerometer data that falls outside the threshold range and the lock is already activated, then at block 914 the processor controls the lock to remain activated since the vehicle is still in motion. At block 916, the autolock timer 573 is reset to zero so that the control unit does not release the mobile device after the first few seconds of driving.

If it has been determined that all of the collected and analyzed accelerometer data falls within the threshold range, then operations may continue at block 918 by determining whether the autolock timer is activated. If the autolock timer has not yet been activated (e.g., the vehicle was just started and not yet been switched from neutral to drive), then operations may revert to the beginning of the routine 900 with the next data from the x-axis, y-axis and z-axis accelerometers being collected and analyzed. Otherwise, if the autolock timer has previously been started, operations may continue at 920 where it is determined whether the autolock timer has reached its limit (e.g., 20 seconds). If the limit has not been reached, operations may revert to the beginning of the routine and more accelerometer data is collected and analyzed to ensure that the data remains within the threshold range for a sufficient amount to time to confidently determine that the vehicle is idle. If the deactivation timer has in fact reached its limit, then at block 922 the lock is deactivated, leaving the driver free to access the mobile device from the control unit casing. Optionally, operations may continue from the beginning of the routine 900 even after the control unit is unlocked until the mobile device is removed from the control unit, the vehicle is turned off, or both.

In the example of the above routines 600, 700 and 900, calibration data is collected every time the driver starts the vehicle. However, in other instances, certain calibration data can be collected less frequently or even never. For instance, the baseline blink rate 532 for a given driver, the driver's baseline eye-closing threshold and eye-opening threshold 534, and the driver's maximum blink duration 536 can be saved in the control unit memory for use the next time the same driver operates the vehicle. Furthermore, the control unit could be programmed to store blink rates, threshold values and maximum blinks for multiple drivers. Alternatively or additionally, calibration routines could be executed every time the driver turns on the vehicle, thereby accumulating data over multiple calibration routines and deriving calibration values from the cumulative data.

In another instance, eye-closing and eye-opening thresholds could be derived directly from data collected during the monitoring stage without reference to calibration data, such as by searching for local maxima and local minima in the collected data and attributing the local maxima and minima with eye-closing and eye-opening events, respectively.

In yet another instance, vehicle motion could be derived directly from accelerometer data collected during the monitoring stage without reference to calibration data, such as by integrating the acceleration data from an x-axis accelerometer (forward-back direction of the vehicle) over time to obtain a snapshot of the vehicle's instant speed at a given moment. In such an instance, when the vehicle's speed is determined to exceed 0 MPH by a preset threshold error margin, the processor may activate the lock and initiate the autolock timer, and when the vehicle's speed is within the threshold error margin of 0 MPH for the duration of autolock timer, the processor may deactivate the lock.

In some instances, the calibration routine could be replaced by preset data, such as standard vibration threshold ranges for a given make and model vehicle.

The above examples generally describe a system for monitoring an operator of an automotive vehicle. However, those skilled in the art will readily appreciate that the underlying principles of the present disclosure are similarly applicable to other vehicles or machines, including but not limited to trains, busses, aircraft, watercraft, construction machinery, and so on. In fact, the underlying principles for preventing an operator's access to a device can be applied to any machine having a motor or generating detectable vibrations. Moreover, the underlying principles for monitoring movements of an operator's eyes can be applied to any task performed by any operator, even for machines that do not have a motor and that do not generate detectable vibrations. Accordingly, those skilled in the art will recognize the broad applicability of the present application to other tasks requiring an operator's focus and attention, as well as to other tasks performed by an operator using a machine having a motor.

The above examples also generally describe preventing the operator from accessing a mobile device, such as a smartphone or any other mobile device that can divert a user's or operator's attention, such as a wearable device (e.g., pager, smartwatch, etc.). However, those skilled in the art will appreciate that the underlying principle of preventing diversions in the present disclosure is equally applicable to software capable of distracting the operator, such as a software module built into the machine being operated. For example, in the case of a vehicle, the routines described in the present application can be used to lock or disable various consoles built into the operator's vehicle, such as an entertainment console or a navigation console. In such cases, instead of physically locking the operator's mobile device, the monitoring routine may functionally lock access to the potentially distracting console (e.g., change the radio station, enter a new navigation destination, etc.) while the vehicle is moving, and not unlock access to the console until it is sufficiently determined that the vehicle is no longer moving.

In a similar vein, the above examples generally describe a control unit having a casing separate from the body of the machine being operated, such as a cradle for holding a smartphone. However, as those skilled will recognize the applicability of the present disclosure's routines to locking and disabling built-in software modules of the machine, it will also be recognized that the instructions of the control unit can be programmed into the machine itself. Thus, in the case of locking or disabling a built-in console or program of the machine being operated, the control unit may use a communications module (e.g., Bluetooth receiver) of the machine to receive signals from the data acquisition unit and to control access to the machine's software based on the received signals in accordance with the routines described above.

Furthermore, some aspects of the disclosed routines can be applicable in settings even outside of machine operation. For instance, worker productivity could be generally tracked using the monitoring routines of the present disclosure. The worker could wear a data acquisition unit while working, and signals from the data acquisition unit could be transmitted to a control unit in the worker's facility for processing. Similarly, a lockable casing could be provided for storing the worker's mobile device. In one such instance, the signals from the data acquisition unit could be used to track an amount of time for which the worker is productive or unproductive and the casing could be programmed to unlock after the worker reaches a predetermined amount of productive time.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A control unit for monitoring attentiveness of an operator, comprising:
a wireless receiver configured to receive digital signals indicative of electrical activity induced by ion flow in the operator's head as a result of the operator blinking; and
a control circuit communicatively coupled to the wireless receiver and configured to:
identify a plurality of eye-closing events and a plurality of eye-opening events based on the received digital signal; and
determine a state of attentiveness of the operator based on the identified eye-closing and eye-opening events.

2. The control unit of claim 1, wherein the control unit is adapted to be mounted to a machine being operated by the operator, and further comprises one or more accelerometers configured to detect a magnitude of acceleration of the machine, and wherein the control circuit is further configured to:
receive the detected magnitude of acceleration from the one or more accelerometers; and
determine whether the machine is in motion or idle based on the detected magnitude of acceleration.

3. The control unit of claim 2, wherein the control unit further comprises:
a housing adapted to house the control circuit and an alarm configured to notify the operator of a determination that the operator lacks attentiveness, and further adapted to encase a mobile device of the operator; and
a solenoid lock adapted to lock the housing, wherein the mobile device cannot be accessed by the operator when the mobile device is encased in the housing and the housing is locked by the solenoid lock,
wherein the control circuit is further configured to actuate a bolt to lock the housing when the machine is determined to be in motion.

4. The control unit of claim 3, wherein the housing further comprises:
a back casing, wherein the bolt is mounted to the back casing;
a hinged front panel; and
a slot adapted to receive the bolt and mounted to the front casing, wherein actuating the bolt causes the bolt to be inserted into the slot, thereby locking the hinged front panel to the back casing.

5. The control unit of claim 3, wherein the control unit further comprises a pressure sensor configured to sense a presence of the mobile device in the housing, wherein the control circuit is configured to receive an indication from the pressure sensor that the mobile device is in the housing and to actuate the bolt when the mobile device is in the housing and the machine is in motion.

6. A method for monitoring attention of a machine operator, the method executed by a processor and comprising:
receiving data from a capacitive electrode positioned in proximity to the operator's head to detect an ion flow of electrical impulses between the driver's brain and eyelids;
identifying a plurality of first events from the received data, each first event corresponding to the operator's eyes closing;
for each identified first event, identifying a second event in the received data following the first event, the second event corresponding to the operator's eyes opening;
based on the identified first and second events in the received data, determining a lack of attentiveness of the operator.

7. The method of claim 6, wherein the ion flow being greater than or equal to a maximum threshold value is indicative of one of a first event or a second event, and the magnitude of electrical activity being less than or equal to a minimum threshold value is indicative of the other of the first event or second event.

8. The method of claim 6, further comprising, for each pair of first and second events, comparing an interval between the first and second events to a maximum blink duration value, wherein the interval exceeding the maximum blink duration value is indicative of a lack of attentiveness of the operator.

9. The method of claim 8, wherein the method comprises a calibration stage and a subsequent monitoring stage, the calibration stage comprising:
receiving data from the capacitive electrode for a preset calibration duration;
for each pair of first and second events identified in the data received during the calibration duration, calculating an interval between the first and second events; and
selecting the maximum interval from among the calculated intervals as the maximum blink duration value,
and the monitoring stage comprising:
receiving data from the capacitive electrode;
identifying a first event from the data received during the monitoring stage, the first event corresponding to the operator's eyes closing;
identifying a second event from the data received during the monitoring stage, the second event corresponding to the operator's eyes opening; and
comparing an interval between the first and second events of the monitoring stage to the maximum blink duration value.

10. The method of claim 6, further comprising:
receiving data from the capacitive electrode for a preset duration during a monitoring stage;
for each pair of first and second events identified in the data received during the duration of the monitoring stage, identifying a blink of the operator;
calculating a blink rate of the operator over the duration of the monitoring stage based on the identified blinks;
comparing the calculated blink rate to each of a maximum blink rate value and minimum blink rate value, wherein the measured blink rate being greater than the maximum blink rate value or less than the minimum blink rate value in indicative of a lack of attentiveness of the operator.

11. The method of claim 10, wherein the method comprises a calibration stage prior to the monitoring stage, the calibration stage comprising:
collecting a set of received data over a preset calibration timespan; and
calculating a baseline blink rate of the operator over the preset calibration timespan, wherein the maximum blink rate value is derived from the baseline blink rate and indicates that the operator is engaged in a conversation, and wherein the minimum blink rate value is derived from the baseline blink rate and indicates that the operator is reading.

12. The method of claim 11, wherein the maximum blink rate value is at least 1.88 times greater than the baseline blink rate.

13. The method of claim 11, wherein the minimum blink rate value is at most 0.55 times the baseline blink rate.

14. The method of claim 6, further comprising, in response to determining a lack of attentiveness, at the processor, one of activating an alarm and logging a lack of attentiveness event in a datalog.

15. A method for preventing distraction to a machine operator, the method executed by a processor and comprising:
upon receiving an indication of the machine being activated, initiating a calibration stage;
during the calibration stage, collecting data from one or more accelerometers, each accelerometer indicating a magnitude of acceleration along a respective axis of the machine;
for each accelerometer, based on the collected data during the calibration stage, calculating a threshold range of acceleration values;
after the calibration stage, initiating a monitoring stage;
during the monitoring stage:
collecting data from the one or more accelerometers;
for each accelerometer, comparing the data collected from the accelerometer during the monitoring stage to the accelerator's calculated threshold range; and
if the data collected from an accelerator during the monitoring stage falls outside the corresponding threshold range, activating a lock to prevent distraction to the machine operator.

16. The method of claim 15, wherein each of the one or more accelerometers has a sensitivity of at least 10 mV/g.

17. The method of claim 15, wherein the threshold range of acceleration values corresponds to a vibration of the machine when the machine is powered and stationary, and wherein when the machine being not powered or in motion results in acceleration values outside of the threshold range.

18. The method of claim 15, wherein the one or more accelerometers comprises:
a first accelerator measuring acceleration along an up-down axis of the machine;
a second accelerator measuring acceleration along a left-right axis of the machine; and
a third accelerator measuring acceleration along a front-back axis of the machine.

19. The method of claim 15 further comprising:
in response to activating the lock, continuing the monitoring stage; and
if the data collected from each accelerator during the continued monitoring stage remains within the corresponding threshold range for a predetermined duration, deactivating the lock.

20. The method of claim 16, wherein the machine is an automotive vehicle, and wherein activating the lock comprises locking a mobile device belonging to the operator in a casing, and wherein deactivating the lock permits the operator to retrieve the mobile device from the casing.
